(19) United States
(12) Patent Application Publication
(10) Pub. No.: US 2005/0158368 A1
(43) Pub. Date: Jul. 21, 2005

(54) Bathing article having detergent contained in a surface layer

(76) Inventor: Connie Hsu, Taipei City (TW)

Correspondence Address: BIRCH STEWART KOLASCH & BIRCH, PO BOX 747, FALLS CHURCH, VA, 22040-0747, US

(21) Appl. No.: 10/759,429

(22) Filed: Jan. 20, 2004

(51) Int. Cl. A61K 9/70

(52) U.S. Cl. 424/443

(57) ABSTRACT

A bathing article having detergent contained on a surface layer for disposable use includes a bathing substance which has a porous structure and a thin layer of detergent or thin coating of soap on the surface of the bathing substance preferably by spraying. It is ready to use after wetting. The quantity of the detergent or soap layer is for use only about one time. Thus it is disposable and especially desirable for one time consumption and hygiene-conscious occasions (such as restaurants and hotels).

### FIELD OF THE INVENTION

The present invention relates to a bathing article having detergent contained in a surface layer and particularly to a disposable mesh, fibrous or sponge type bathing substance with the detergent sprayed to a surface layer.

### BACKGROUND OF THE INVENTION

Conventional bathing substances and detergents are usually made and stored separately. When in use, the bathing substance is wetted, and then the detergent is added. After the bathing substance is squeezed and scrubbed to generate a frothy lather, it may be used to scrub the body for cleaning. It is a troublesome chore.

To overcome this, there is an approach to make the detergent in a cake as disclosed in ROC patent publication No. 172897, entitled “Tearable fibrous soap” which has the detergent mixed in a fibrous bathing substance as shown in FIG. 1. It is more convenient than the conventional ones. As it already contains detergent, and is ready for use without the need to add detergent. Such a fibrous soap has filaments jutting slightly out to clear away dirt and massage the body. There is no need to waste time fetching detergent and repeatedly scrubbing. When the soap is consumed it becomes thin and small. The filaments extend outside; these fibers may be removed to prevent the filaments from extending too far and hindering the contact between soap and skin.

While it is more convenient, it is still not desirable for use in hotels. People are very concern of personal hygiene these days; hotels usually provide personal bathing articles for guests who stay over a short period. The conventional cleaning article mentioned above contains a lot of detergent. However, it has to be thrown away after use for one or a few times for hygienic reasons, regardless if there is still detergent. It is wasteful.

### SUMMARY OF THE INVENTION

Therefore the primary object of the invention is to provide a disposable bathing article that has detergent contained in a surface layer to meet personal hygienic requirements. It includes a bathing substance and detergent. The bathing substance is made from a mesh material, porous fibers or sponge. A thin layer of detergent or soap film is coated on the surface layer of the porous material. The detergent or soap film is preferably applied through spraying.

The foregoing, as well as additional objects, features and advantages of the invention will be more readily apparent from the following detailed description, which proceeds with reference to the accompanying drawings.

### BRIEF DESCRIPTION OF THE DRAWINGS

FIG. 1 is a schematic view of a conventional bathing article.

FIG. 2 is a perspective view of the present invention for surface processing.

FIG. 3 is a schematic view of an embodiment of the present invention.

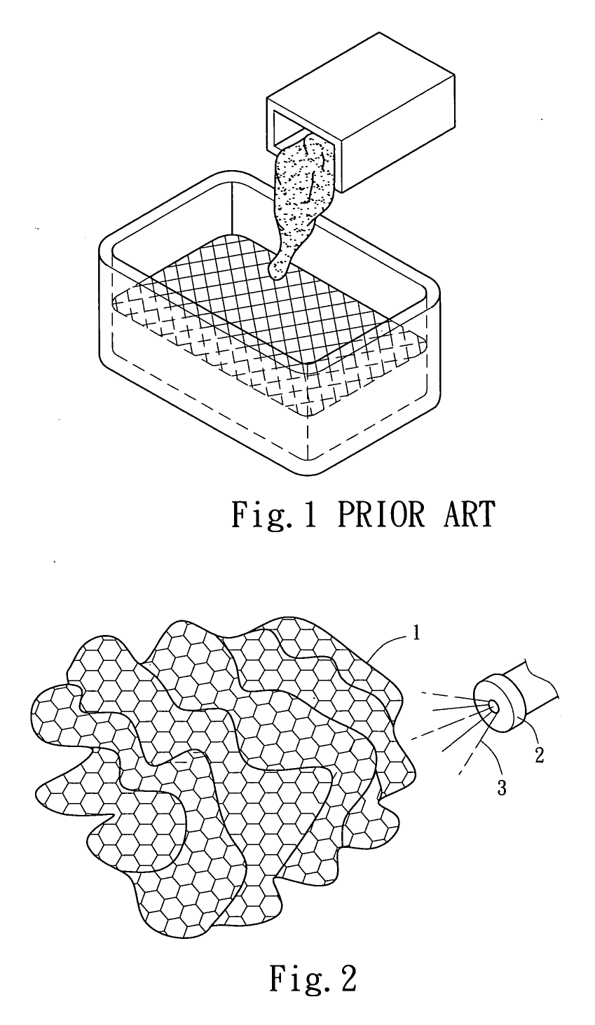

### DETAILED DESCRIPTION OF THE PREFERRED EMBODIMENTS

Referring to FIG. 2, the bathing substance 1 according to the invention includes a porous structure coated with a thin layer of detergent 3 or soap on the surface. The detergent 3 may be ejected from a nozzle 2 to adhere to the surface of a bathing substance 1. As the detergent 3 is coated on the surface of the bathing substance 1 by spraying, the bathing substance 1 has to be made from a material of a lower density such as a mesh material, porous fibrous material or sponge so that the detergent 3 may attach more easily.

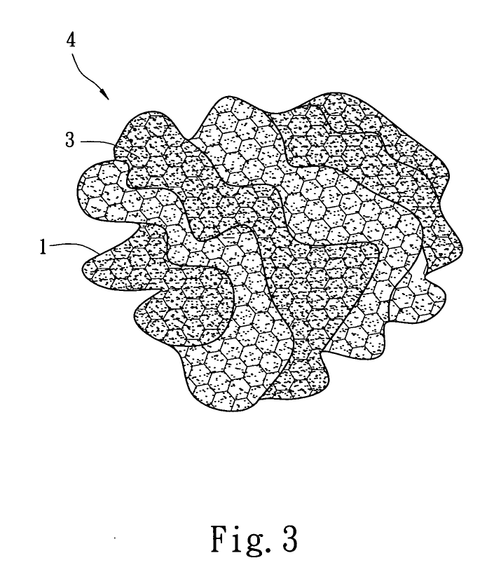

Refer to FIG. 3 for a bathing article 4 that contains detergent 3 according to the invention. As the bathing substance 1 has detergent 3 adhered on the surface, once wetted, it may be used to clean the body. Since the detergent 3 is attached merely to the surface of the bathing substance 1, its may be used about once. Thus the bathing article 4 of the invention may be disposable.

While the preferred embodiments of the invention have been set forth for the purpose of disclosure, modifications of the disclosed embodiments of the invention as well as other embodiments thereof may occur to those skilled in the art. Accordingly, the appended claims are intended to cover all embodiments which do not depart from the spirit and scope of the invention.

1. A bathing article having detergent contained in a surface layer comprising a bathing substance and a detergent to couple with said bathing substance, said bathing substance being made from a porous material for scrubbing and cleaning the skin, said detergent being coated on the surface of said bathing substance by spraying through a nozzle.

2. The bathing article of claim 1, wherein said bathing substance is made from a mesh material.

3. The bathing article of claim 1, wherein said bathing substance is made from porous fibers.

4. The bathing article of claim 1, wherein said bathing substance is made from sponge.

5. The bathing article of claim 4, wherein said sponge is a natural or man-made.

6. The bathing article of claim 1, wherein said detergent is a film of soap.
